(19) United States
(12) Patent Application Publication
(10) Pub. No.: US 2008/0008747 A1
(43) Pub. Date: Jan. 10, 2008

(54) Transdermal patch

(76) Inventor: Robert B. Royds, Plainsboro, NJ (US)

Correspondence Address: Kenneth Watov, Esq.;WATOV & KIPNES, P.C., P.O. Box 247, Princeton Juncton, NJ, 08550, US

(21) Appl. No.: 11/482,206

(22) Filed: Jul. 7, 2006

(51) Int. Cl. A61K 9/70

(52) U.S. Cl. 424/449

(57) ABSTRACT

A transdermal patch for administrating an agonist to a patient, where the transdermal patch includes an occlusive wall defining a reservoir with an open bottom end and an opposing top end, a matrix permeable to perspiration from the patient occupying the reservoir, wherein the matrix includes a first region having the agonist suspended therein for release through the bottom end of the reservoir to the patient, and a second region located at the top end of the reservoir, wherein the second region includes an antagonist associated with the agonist suspended therein and being configured to release the antagonist at the onset of imminent overdose of the agonist by the patient, a permeable adhesive layer covering at least a portion of the bottom end of the reservoir, the adhesive layer being adapted for maintaining the matrix in communication with the skin of the patient; and at least one visual indicator located at the top end of the reservoir, wherein the at least one visual indicator is adapted to undergo a visual change in the presence of the perspiration from the patient.

### RELATED APPLICATION

This application is related to Ser. No. 11/333,602, filed on Jan. 17, 2006, for “Abuse Resistant Transdermal Drug Delivery,” the teachings of which are incorporated herein by reference to the extent they do not conflict herewith.

### FIELD OF THE INVENTION

The present invention relates to drug delivery systems, and more particularly to a transdermal drug delivery system in the form of a transdermal patch.

### BACKGROUND OF THE INVENTION

Transdermal drug delivery systems have been developed for administration and delivery of pharmaceuticals including therapeutic agents at desired sustained levels by absorption through the skin. Such systems are typically embodied in the form of a transdermal patch, and offer advantages, which are not readily achievable by other modes of administration. The transdermal patch is a medicated adhesive patch that is placed on the skin to deliver a sustained- or time-released dose of medication through the skin and into the bloodstream. Transdermal patches are used to deliver a wide variety of pharmaceuticals.

One widely used type of transdermal patch is the “matrix” type, which generally includes a backing material, a drug reservoir, and an adhesive. The backing material is inert to the pharmaceutical or drug formulation contained in the patch, and prevents migration of the pharmaceutical. The drug reservoir is a matrix in which the pharmaceutical is dispersed and through which it migrates by diffusion or microporous flow. The matrix material may simultaneously act as an adhesive as well, in which case only an occlusive, removable covering or liner is required to complete the system. The transdermal patch provides a relatively simple dosage regimen, and it also provides a relatively slow and controlled route for release of the pharmaceutical into the systemic circulation.

The transdermal patch possesses some limitations including determining when it is time to change the patch for a fresh one or when a possible overdosing is about to occur. Dosing of any medication by almost any route of administration, has largely been one of “approximation” and “trial and error.” This is especially so with respect to ambulatory patients and long term medication. For this reason, there is a constant need to ensure that the pharmaceutical administered by the transdermal patch is implemented safely and effectively.

Accordingly, there is a need for a transdermal patch designed to deliver a pharmaceutical through the skin and into the circulatory system. There is a further need for a transdermal patch that continuously monitors the proper functioning of the patch as intended. There is a further need for a transdermal patch that is designed to recognize and indicate when a patch is not functioning properly, when the supply of pharmaceutical has been exhausted, or when an overdose is imminent in order to prevent or halt such overdose event.

### SUMMARY OF THE INVENTION

The present invention relates generally to a transdermal drug delivery system in the form of a transdermal patch. The transdermal patch of the present invention is adapted to deliver a therapeutic agent generally in the form of an agonist such as an opioid to the patient. In accordance with the present invention, the transdermal patch is adhesively applied to the skin of the patient. Perspiration containing moisture, ions, electrolytes, and other secretions, will diffuse into the patch in a controlled manner, while the therapeutic agent migrates from the patch into the patient through the skin at a predictable rate, according to corresponding gradient forces. The transdermal patch of the present invention includes safeguards to prevent tampering that may lead to abuse, and to prevent problems associated with imminent overdosing by the patient. Furthermore, the transdermal patch of the present invention includes visually perceptible indicating means to keep the patient informed about the operating status of the patch.

Preferably, the transdermal patch of the present invention includes a therapeutic agent containing matrix composed of first and second regions. The first region includes the therapeutic agent in the form of an agonist dispersed therein, and the second region includes an antagonist to the therapeutic agent dispersed therein. The patient has increased tendency of experiencing an overdose episode when the transdermal patch remains on the patient's skin for an excessively prolonged period of time. In such an event, the transdermal patch of the present invention is configured to release a corresponding antagonist at the proper time to neutralize the effects of the agonist, thereby ensuring that the patient avoids life-threatening toxicity or adverse effects related to an overdose. Alternatively, an overdose can occur due to a patient's skin characteristics facilitating a relatively more rapid absorption of the agonist, whereby the present invention's subsequent delivery of the antagonist provides safeguards against the effects of the agonist, thereby protecting the patient from a dangerous overdose.

More preferably, the transdermal patch of the present invention further includes a visual indicator located at the top end of the reservoir, wherein the visual indicator is adapted to undergo a visual change in the presence of the perspiration from the patient. Perspiration containing moisture and electrolytes can readily diffuse into the present patch, which at a pre-determined time based on the corresponding diffusion rate, reaches the indicator to effect the visual change. In this manner, the visual indicator can thereby be adapted to inform the patient about the operating status of the transdermal patch.

The present invention includes the description of a visual indicator that can be designed, through its formulation, to effect a visible change at significant time points in the lifetime of the patch. Since the dynamics of the mechanism effecting the visual change or changes are associated with the release of the drug (i.e., agonist), the configuration of the present patch can be tailored to provide visual indicators representing the status of the drug release from the matrix. For example, the release of a sufficient drug quantity to exert a therapeutic action can be associated with one color change indicator, and the near exhaustion of drug reserves from the matrix or potential imminent overdose in the patient, can be associated with a second color change. This second feature, in particular, will serve as an indicator to the patient that the current patch should be removed and discarded.

In one aspect of the present invention, there is provided a transdermal patch for administrating an agonist to a patient, where the transdermal patch comprises:

a) an occlusive wall defining a reservoir with an open bottom end and an opposing top end;

b) a matrix occupying the reservoir, the matrix comprising:
a first region having the agonist suspended therein for release through the bottom end of the reservoir to the patient; and
a second region having an antagonist associated with the agonist suspended therein and being configured to release the antagonist at a predetermined time after the initial release of the agonist from the first region; and

c) a permeable adhesive layer covering at least a portion of the bottom end of the reservoir, the adhesive layer being adapted for maintaining the matrix in communication with the skin of the patient.

Preferably, the second region is configured to release the antagonist in the event of imminent overdose of the agonist released from the first region.

In a further aspect of the present invention, there is provided a transdermal patch for administrating an agonist to a patient, where the transdermal patch comprises:

a) an occlusive wall defining a reservoir with a bottom end and an opposing top end;

b) a matrix permeable to perspiration from the patient occupying the reservoir, the matrix comprising:
a first region having the agonist suspended therein for release through the bottom end of the reservoir to the patient; and
a second region located at the top end of the reservoir, the second region having an antagonist associated with the agonist suspended therein and being configured to release the antagonist at the onset of imminent overdose of the agonist by the patient;

c) a permeable adhesive layer covering at least a portion of the bottom end of the reservoir, the adhesive layer being adapted for maintaining the matrix in communication with the skin of the patient; and

d) at least one visual indicator located at the top end of the reservoir, the at least one visual indicator being adapted to undergo a visual change upon contact with the perspiration from the patient.

In an even further aspect of the present invention, there is provided a transdermal patch for administrating an agonist to a patient, where the transdermal patch comprises:

a) an occlusive wall defining a reservoir with a bottom end and an opposing top end;

b) a matrix permeable to perspiration from the patient occupying the reservoir, the matrix comprising:
a first region having the agonist suspended therein for release through the bottom end of the reservoir to the patient; and
a second region located at the top end of the reservoir, the second region having an antagonist associated with the agonist suspended therein and being configured to release the antagonist at the onset of imminent overdose of the agonist by the patient;

c) a permeable adhesive layer covering at least a portion of said bottom end of the reservoir, the adhesive layer being adapted for maintaining the matrix in communication with the skin of the patient; and

d) primary, first, second and third visual indicators each located at the top end of the reservoir, the primary, first, second and third visual indicators each being adapted to undergo a visual color change upon contact with the perspiration from the patient.

### BRIEF DESCRIPTION OF THE DRAWINGS

The following drawings, in which like items may have the same reference designations, are illustrative of embodiments of the present invention and are not intended to limit the invention as encompassed by the claims forming part of the application, wherein:

FIG. 1 is cross-sectional view of a transdermal patch for one embodiment of the present invention; and

FIG. 2 is a cross-sectional view of a transdermal patch for another embodiment of the present invention.

### DETAILED DESCRIPTION OF THE INVENTION

The present invention is directed generally to a transdermal drug delivery system in the form of a transdermal patch. The transdermal patch of the present invention is adapted to deliver a therapeutic agent generally in the form of an agonist such as an opioid to the patient. In accordance with the present invention, the transdermal patch is adhesively applied to the skin of the patient. Perspiration containing moisture, ions, electrolytes, and other secretions, will diffuse into the patch in a controlled manner, while the therapeutic agent migrates from the patch into the patient through the skin, according to corresponding gradient forces. The transdermal patch of the present invention includes safeguards to prevent tampering that may lead to abuse, and to prevent problems related to overdosing by the patient. The transdermal patch of the present invention is specifically constructed to prevent illicit diversion of the opioid agonist for non-medical or non-therapeutic use. Furthermore, the transdermal patch of the present invention includes visually perceptible indicating means to keep the patient informed about the operating status of the patch.

In accordance with the present invention, the transdermal patch includes a matrix having a first region with a therapeutic agent, preferably an agonist, and more preferably an opioid agonist, dispersed therein, and a second region with an antagonist capable of neutralizing the pharmacological effects of the therapeutic agent in the patient's body, dispersed therein. Each of the regions is formulated and positioned to release its respective contents under different timing circumstances and conditions during usage. This arrangement provides an effective mechanism to substantially minimize or prevent overdosing when the transdermal patch remains on the patient's skin for an excessively prolonged period of time. The latter condition may occur especially in elderly and ambulatory patients. This arrangement provides a further mechanism to prevent unacceptable tampering that may lead to abuse. The agonist and antagonist are retained in the present patch in a manner, which effectively restricts the user's ability to illicitly extract the agonist without contamination by the antagonist. Accordingly, this combination of illicit diversion prevention and overdose protection yields a drug delivery system with an enhanced safety profile and therapeutic effectiveness, while at least maintaining or preserving the efficacy of the administered agonist.

In a general embodiment of the present invention, there is provided a transdermal patch for administrating an agonist to a patient. The transdermal patch includes an occlusive wall defining a reservoir with an open bottom end and an opposing top end, and a matrix occupying the reservoir. The matrix includes a first region having the agonist suspended therein for release through the bottom end of the reservoir, and a second region including an antagonist associated with the agonist suspended therein. The second region is configured to release the antagonist at a predetermined time after the initial release of the agonist from the first region of the matrix. The transdermal patch further includes a permeable adhesive layer covering at least a portion of the bottom end of the reservoir, and the adhesive layer being adapted for maintaining the matrix in communication with the skin of the patient.

In accordance with the present invention, the consistencies and the migration characteristics of the matrix and its regions can be altered or modified to provided different delivery times and rates depending on the agonist and antagonist combination. In addition, the concentrations of the agonist and antagonist can be varied to delivery different doses, rates and potencies.

Preferably, the transdermal patch of the present invention further includes a visual indicator located at the top end of the reservoir, wherein the visual indicator is adapted to undergo a visual change in the presence of the perspiration from the patient. Perspiration containing moisture and electrolytes can readily diffuse into the present patch, which at a pre-determined time based on the corresponding diffusion rate, reaches the indicator to effect the visual change. In this manner, the visual indicator can thereby be adapted to inform the patient about the operating status of the transdermal patch.

The present invention includes the description of a visual indicator that can be designed, through its formulation, to effect a visible change at significant time points in the lifetime of the patch. Since the dynamics of the mechanism effecting the visual change or changes are associated with the release of the drug (i.e., agonist), the configuration of the present patch can be tailored to provide visual indicators representing the status of the drug release from the matrix. For example, the release of a sufficient drug quantity to exert a therapeutic action can be associated with one color change indicator, and the near exhaustion of drug reserves from the matrix or potential imminent overdose in the patient, can be associated with a second color change. This second feature, in particular, will serve as an indicator to the patient that the current patch should be removed and discarded.

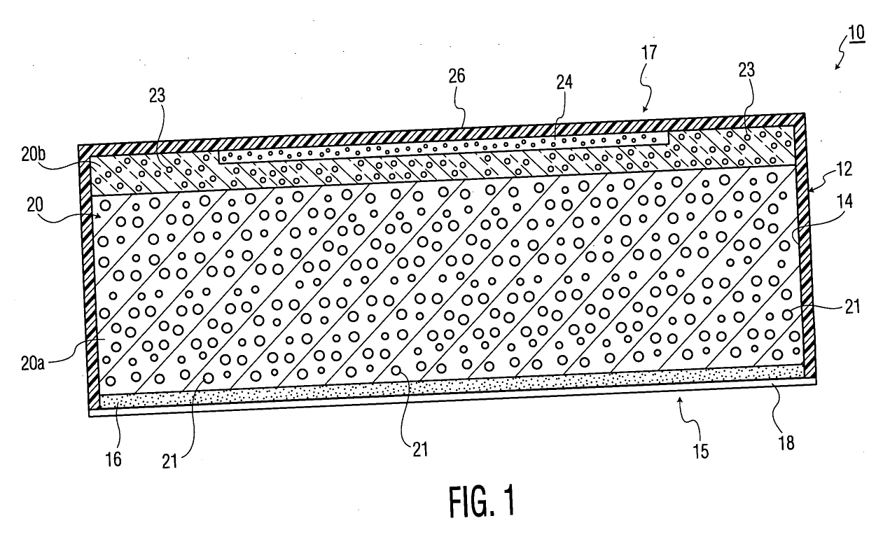

Referring to FIG. 1, there is shown a cross-sectional view of a transdermal patch, designated generally by the reference 10 for one embodiment of the present invention. The patch 10 comprises a translucent occlusive wall, or backing layer 12 with a top face portion 26. The wall 12 provides a reservoir 14 having an open bottom end 15 and an opposing top end 17. The wall 12 can be composed of a medically approved plastic material including plastic composites formed by any suitable technique. Other suitable materials, generally of plastic polymeric composition, can be used for the wall 12, and are known to those skilled in the art. The reservoir 14 includes a matrix 20, which is formulated to absorb several times its own weight in water, and capable of suspending a drug for subsequent release.

In a preferred embodiment of the present invention, the matrix 20 may be composed of guar, acacia, or xanathan gum, or a gelling agent or polymer such as carboxypolymethylene, hydroxyethylcellulose or polyacrylamide. In the case of guar gum, for example, the matrix 20 can be formulated to absorb from about 5 to 10 times its own weight. The matrix 20 includes a first region 20a and a second region 20b located at the top end 17 of the reservoir 14. The first and second regions 20a and 20b can be composed of the same material or different materials exhibiting different diffusion rates and/or chemical properties. The sensitivity of the matrix material to the permeation of moisture is controlled by the choice of materials or formulation. The matrix 20 is designed to allow moisture, ions, electrolytes and the like, typically, present in perspiration, to diffuse or permeate in order to release the therapeutic agent for delivery to, and subsequent passage through the skin of the user as will be described hereinafter.

The first region 20a of the matrix 20 comprises a therapeutic agent 21, preferably an agonist, suspended therein. The agonist can be an opioid agonist useful for treating or preventing a disease, condition or symptoms thereof including alleviation of pain in a warm-blooded animal including a human. The second region 20b of the matrix 20 comprises an antagonist 23 to the therapeutic agent 21, which is a pharmaceutical agent that inhibits or blocks the biologically active effects of the agonist 21 contained in the first region 20a. The first and second matrix regions 20a and 20b are suitably formulated and positioned with one another to provide differential rates and times of delivery, while obstructing tampering for illicit diversion as will be further described hereinafter.

The term “opioid agonist” is defined for purposes of the present invention to mean any opioid-based compound including opioid peptides, opium alkaloids, semi-synthetic and fully synthetic opioids, capable of binding to an opioid receptor and triggering a response in a cell, and include bimodally acting opioid agonists. The term “opioid agonist” can be used interchangeably with the term “opioid.”

Suitable examples of opioid agonists 21 useful in the present invention, include, but are not limited to, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propoxyphene, sufentanil, tilidine, tramadol, combinations thereof, salts thereof, and the like.

Preferred examples include hydrocodone, morphine, hydromorphone, oxycodone, codeine, levorphanol, meperidine, methadone, salts thereof, and combinations thereof.

The term “opioid antagonist” is defined for purposes of the present invention to mean any opioid-based compound capable of binding to the same opioid receptor of a corresponding opioid agonist, and preventing or blocking the activation of the receptor. Suitable examples of opioid antagonist 23 useful in the present invention include naltrexone, nalmefene, cyclazacine, levallorphan and mixtures thereof. Preferably, the opioid antagonist is naloxone or naltrexone.

Referring back to FIG. 1, the matrix 20 is maintained in contact with the patient's skin during administration via a permeable adhesive layer 16 in one embodiment of the present invention. The permeable adhesive layer 16 can be composed of a suitable pressure-sensitive adhesive material, and is located at the bottom end 15 of the reservoir 14 overlaying the bottom portion of the matrix 20. The adhesive layer 16 enables the matrix 20 and the wall 12 to be secured to the skin of the patient, while permitting free passage of molecules (e.g., perspiration and drug) in between the patch 10 and the patient's skin. It will be understood that when the patch 10 is provided to the patient, the adhesive layer 16 is normally covered with a disposable protective layer 18 that the patient must remove prior to application. When attached to the skin of the patient, the wall 12 provides an occlusive covering, which enhances hydration of the skin area covered by the patch 10, and diffusion of the perspiration into the matrix 20. Hydration of the skin fosters release and absorption of the drug associated with the patch 20. In another embodiment of the present invention, substantial portions of the adhesive layer 16 can be removed or eliminated to provide direct contact of the matrix 20 with the patient's skin for enhancing ease of delivery.

In a preferred embodiment of the present invention, the patch 10 further includes a visual indicator 24 within the top end 17 of the reservoir 14. The visual indicator 24 is adjacent to and operatively associated with the top face portion 26 of the wall 17, and visible through the wall 12. Preferably, the visual indicator 24 is a microencapsulated color indicator, wherein the color agent is encapsulated within a coating material. The coating material may be selected from arylate resins or methylmetacrylic acid co-polymers, or from formulations of hydrophilic ethylcellulose derivatives and hydrophobic methylcellulose derivatives.

The indicator 24 is adapted to change color in response to the presence of water, moisture, electrolyte, ions, or other secretions present in perspiration, and can be produced from inorganic salts, which change color upon hydration such as, for example, anhydrous copper sulfate or cobalt chloride. In an alternative embodiment of the present invention, colorful dyes such as amaranth or mercurochrome can be microencapsulated to effect a color change when released. The microencapsulation can be formulated for selective timing of the activation of the color change in the presence of water, moisture, electrolyte, ion or other secretion. This enables the indicator 24 to be tailored to accurately reflect the status of drug release from the patch 10, either by an appropriate choice of coating material or by manipulation of the components in the matrix 20. This feature is advantageous in instances where the timing of events such as the onset, peak, decline and end of the therapeutic delivery of the drug is an important consideration in the proper use of the patch 10. Indicator 24 can be provided by any indicator which reacts to changes in ion concentration about or near the physiological range, for example, erythrolimin, bromothyol blue, neutral red, phenol red, thymol blue, phenolthalein or other appropriate acid/base indicators.

The material of the matrix regions 20a and 20b can be selected or formulated to control of the rate of drug release from the matrix 20, as well as the diffusion rate of the perspiration through which the corresponding matrix regions 20a and 20b are activated for release of their associated pharmaceutical agent such as agonist 21 and antagonist 23, respectively. The matrix regions 20a or 20b can be formulated to be relatively impervious to moisture, for example, one that is thicker or less permeable because of its physico-chemical properties, or one that contains a higher content of hydrophobic elements in its composition, will result in a more gradual drug release over a sustained time period and gradual diffusion of the patient's perspiration therethrough. In contrast, a matrix region 20a or 20b that is relatively permeable to water will rapidly release the drug over a relatively shorter time period.

Once the patch 10 is properly applied to the patient's skin, the occlusive wall 12 entraps the patient's perspiration produced from the covered area of the skin. The perspiration permeates through the adhesive layer 16 into the first matrix region 20a in one embodiment, or directly thereinto in an alternative embodiment of the present invention. As the perspiration solvates the matrix material, the suspended agonist 21 is released and flows to the patient's skin for delivery. The diffusion rate of the perspiration through the first matrix region 20a is selected to provide adequate time for administering a full dose of agonist 21 to the patient.

As the perspiration flows into the second matrix region 20b, the antagonist 23 is released therefrom and begins to diffuse through the matrix 20 toward the patient's skin. Eventually, the perspiration reaches the visual indicator 24 and activates a visual change to notify the patient that the patch 10 has delivered the requisite dose amount and to remove the patch 10 to prevent an overdose. The patient is provided a short time to remove the patch 10 as the antagonist diffuses or migrates through the matrix 20. If the patient does not remove the patch 10, the antagonist 23 is subsequently delivered to the patient through the skin. The administration of the antagonist 23 reverses the therapeutic effects of the agonist previously administered, and prevents or avoids any complications that may arise from the imminent drug overdose.

The second matrix region 20b also operates to deter tampering for the purpose of illicitly diverting the agonist 21 contained in the first matrix region 20a. The second matrix region 20b is fragile and physically disruptable, and also soluble in the presence of any solvent that may be used by abusers to extract the agonist from the first matrix region 20a. Accordingly, if an abuser attempts to mechanically extract the agonist 21 from the matrix 20, the antagonist 23 in the second matrix region 20b will likewise be extracted and mixed with the agonist 21. This will counter the expected “high” effect of the agonist 21. Similarly, if the abuser attempts to use a solvent to extract the agonist 21 from the first matrix region 20a, the second matrix region 20b will dissolve, releasing the antagonist 23 along with the extracted agonist 21, thereby foiling the diversion attempt.

The transdermal patch 10, as described previously, includes the matrix 20 with two regions 20a and 20b, each exhibiting individual diffusion characteristics. The matrix 20 urges the therapeutic agent (e.g., opioid agonist 21) to flow towards the patient's skin based on the concentration gradient. Similarly, the perspiration including moisture, ions, electrolytes and other secretions, produced by the patient, flow into the matrix 20. The indicator 24 which can be sensitive to any of the perspiration components is positioned proximate the top end 17 of the reservoir 14. The therapeutic agent, agonist 21 for example, flows to the patient's skin until such time that a permitted maximum dosage has been administered, at which point the antagonist 23 begins to flow toward the skin to prevent or minimize any potential for an overdose. The matrix 20 is designed so that the perspiration reaches the indicator 24 at about the same time the desired dose is administered to the patient. If the indicator 24 is disregarded, then the patch 10 begins to administer the antagonist 23. The arrangement of the agonist 21 and antagonist 23 further limits the potential of extracting the agonist 21 without contamination by the antagonist 23.

The ratio of the agonist 21 to the opioid antagonist 23 in the transdermal patch 10 is such that the effect of the agonist 21 is at least partially blocked when the patch 10 is chewed, crushed or dissolved in a solvent and heated, and then administered orally, intranasally, parenterally or sublingually. Since the transdermal patch 10 of the present invention, when used as instructed, does not substantially release the antagonist 23 when the agonist 21 is adminstered properly in the alotted time, the amount of such antagonist 23 can be varied more widely than if the opioid antagonist 23 is available to be released into the gastrointestinal system upon oral administration. For safety reasons, the amount of the antagonist 23 present must not be harmful to humans even if fully released. The ratio of particular agonist 21 to antagonist 23 can be determined without undue experimentation by one skilled in the art.

In certain embodiments of the present invention, the ratio of the agonist and the antagonist is about 1:1 to about 50:1 by weight, preferably about 1:1 to about 20:1 by weight. In certain preferred embodiments, the ratio is about 1:1 to about 10:1 by weight. In a preferred embodiment of the invention, the agonist comprises an opioid such as oxycodone or hydrocodone and is present in the amount of about 15 mg to 45 mg and the antagonist comprises naltrexone and is present in about 0.5 mg to 5 mg.

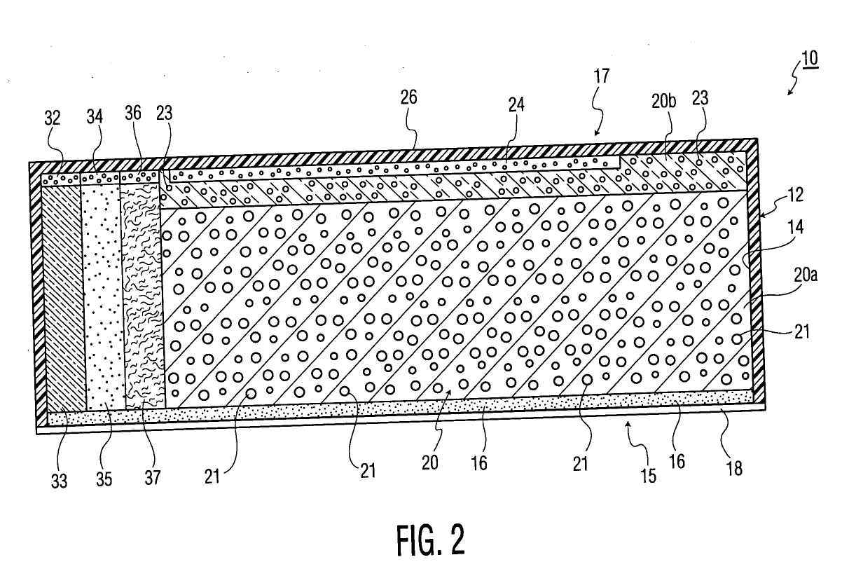

Referring to FIG. 2, a transdermal patch designated generally by reference numeral 30 is shown for an alternative embodiment of the present invention. The embodiment of the patch 30 includes features similar to those described for the transdermal patch 10. The transdermal patch 30 further includes visual indicators 32, 34, and 36 each of which is operatively associated with a corresponding fluid permeable column or timing channels 33, 35, and 37, respectively. The visual indicators 32, 34, and 36 are the same as described for the indicator 24 in the prior embodiment. The timing channels 33, 35, and 37 are configured generally to provide a timing mechanism for the associated visual indicators 32, 34 and 36, respectively. The timing mechanism is implemented by controlling the diffusion rate and distance in which the patient's perspiration travels along the length of the timing channel 33, 35, or 37 from the patient's skin to the corresponding visual indicator 32, 34, or 36, respectively.

The timing channels 33, 35, and 37 are each composed of a fluid passing porous material such as, for example, an adsorbent material capable of conveying moisture, ions, electrolytes, and other secretions from the patient at a predetermined rate of diffusion. The adsorbent material can include, but is not limited to, silica, silica gel, alumina, cellulose, and combinations thereof. The diffusion rate of the adsorbent material can be readily adjusted by varying the porosity, pore size and hydrophobicity of the material as known in the art. The fluid passing porous material can also include materials similar to those used to construct the drug containing matrix 20.

Each of the timing channels 33, 35 or 37 exhibits different rates of diffusion based on the desired timing condition. In this manner, the timing channels 33, 35 and 37 are designed to provide a series of different color indicators to change color at time points corresponding, for example, to the time of onset of drug delivery, the time of peak delivery, the time at which delivery should be discontinued, time when risk of overdose is imminent and time when release of the antagonist begins to prevent the imminent overdose. The color changes that indicate the critical events in the life of the patch 30 can be devised very closely to reflect the true status of drug release from the matrix 20 as will be further described hereinafter.

In one embodiment of the present invention, the visual indicator 32 and the timing channel 33 can be designated to indicate that the patch 30 has been properly applied to the patient's skin and is operating. The adsorbent material used in the timing channel 33 can be formulated to exhibit a high diffusion rate as compared to the matrix 20. Accordingly, the activation of the indicator 32 denotes that the adhesive layer 18 is properly bonded to the patient's skin and that the corresponding delivery of the agonist 21 has been initiated.

The visual indicator 34 and the timing channel 35 can be designated to indicate that the patch 30 is in peak delivery mode of the agonist 21 to the patient. The adsorbent material used in the timing channel 35 can be formulated to exhibit a medium diffusion rate as compared to the matrix 20. Accordingly, the activation of the indicator 34 denotes the peak delivery of the agonist to the patient.

The visual indicator 36 and the timing channel 35 can be designated to indicate that the patch 30 has delivered the desired dose of the agonist 21 to the patient. The adsorbent material used in the timing channel 37 can be formulated to exhibit a slow diffusion rate as compared to the matrix 20. Accordingly, the activation of the indicator 36 denotes the threshold at which the patient is in danger of receiving an overdose of the agonist 21, and that delivery of the antagonist 23 is initiated.

As to the visual indicator 24, the visual change indicates that successful drug delivery has taken place. The indicator 24 ensures compliance to dosing instructions, since the visual change will not be achieved without continued contact with the skin. Observation, therefore, that a visual change did not occur at the expected time in any of the indicators 24, 32, 34, 36, respectively, can prompt further investigation. Therefore, the dermal patch 10 or 30 in accordance with this invention preferably includes at least one indicator, designed to change visually when the drug reserves within the matrix 20 is almost exhausted. The indicator 24 is intended to prompt the user to remove and discard the old patch 10 to avoid imminent overdose of agonist 21, and initiation of the delivery of the antagonist will begin soon after.

The above-described patches 10 and 30 can be used in conjunction with preparatory skin cleanser, containing, for example, alcohol and a weakly buffered acidic or basic solution. The solvent serves to remove surface grease to eliminate a barrier to absorption at the skin, and a buffered acidic or basic solution can be selected according to the physical or chemical properties of the particular drug to be administered, and to maximize drug stability, while enhancing transdermal penetration.

The forgoing discussion discloses and describes merely exemplary embodiments of the present invention. One skilled in the art will readily recognize from such discussion, and from the accompanying claims, that various changes, modifications, and variations can be made therein without departing from the spirit and scope of the invention as defined in the following claims.

What is claimed is:

1. A transdermal patch for administrating an agonist to a patient, said transdermal patch comprising:
a) an occlusive wall defining a reservoir with a bottom end and an opposing top end;
b) a matrix occupying said reservoir, said matrix comprising:
a first region having the agonist suspended therein for release through the bottom end of the reservoir to the patient; and
a second region having an antagonist associated with the agonist suspended therein and being configured to release the antagonist at a predetermined time after the initial release of the agonist from the first region; and
c) a permeable adhesive layer covering at least a portion of said bottom end of the reservoir, said adhesive layer being adapted for maintaining the matrix in communication with the skin of the patient.

2. The transdermal patch of claim 1, wherein the predetermined time is at the onset of imminent overdose of the agonist by the patient.

3. The transdermal patch of claim 1, wherein the first and second regions of the matrix are each adapted to facilitate the diffusion of perspiration therethrough at a predetermined diffusion rate.

4. The transdermal patch of claim 3, wherein the perspiration comprises a member selected from the group consisting of ions, electrolytes, water, moisture, secretions, and combinations thereof.

5. The transdermal patch of claim 4, further comprising at least one visual indicator located at the top end of the reservoir, said visual indicator being adapted to undergo a visual change upon contact with the perspiration from the patient.

6. The transdermal patch of claim 5, wherein the visual change in one of said at least one visual indicator is indicative of the delivery of the complete dose of the agonist to the patient.

7. The transdermal patch of claim 5, wherein the visual change in one of said at least one visual indicator is indicative of the proper bonding of the adhesive layer to the patient's skin.

8. The transdermal patch of claim 5, wherein the visual change in one of said at least one visual indicator is indicative of the peak delivery of the agonist to the patient.

9. The transdermal patch of claim 5, wherein the visual change in one of said at least one visual indicator is indicative of the threshold at which the patient is in danger of receiving an overdose of the agonist.

10. The transdermal patch of claim 5, wherein the at least one visual indicator is a color indicator adapted to irreversibly change color upon contact with the perspiration from the patient.

11. The transdermal patch of claim 10, wherein the at least one visual indicator is a plurality of visual indicators.

12. The transdermal patch of claim 5, comprising a primary visual indicator located proximate the top end of the reservoir in communication with the matrix, said primary visual indicator being adapted to undergo a visual change to indicate delivery of the complete dose of the agonist to the patient.

13. The transdermal patch of claim 4, further comprising at least one fluid permeable column having one of the at least one visual indicator located at one end, and an inlet at the other end proximate the bottom end of the reservoir for receiving the patient's perspiration.

14. The transdermal patch of claim 13, further comprising:
a first fluid permeable column in operative association with a first visual indicator, said first fluid permeable column adapted for facilitating the flow of the perspiration at higher diffusion rate relative to the matrix;
a second fluid permeable column in operative association with a second visual indicator, said second fluid permeable column adapted for facilitating the flow of the perspiration at a similar diffusion rate relative to the matrix; and
a third fluid permeable column in operative association with a third visual indicator, said first fluid permeable column adapted for facilitating the flow of the perspiration at a lower diffusion rate relative to the matrix.

15. The transdermal patch of claim 14, wherein the visual change in the first visual indicator is indicative of the proper bonding of the adhesive layer to the patient's skin.

16. The transdermal patch of claim 14, wherein the visual change in the second visual indicator is indicative of the peak delivery of the agonist to the patient.

17. The transdermal patch of claim 15, wherein the visual change in third visual indicator is indicative of the threshold at which the patient is in danger of receiving an overdose of the agonist.

18. The transdermal patch of claim 13, wherein the at least one fluid permeable column comprises a fluid passing porous material.

19. The transdermal patch of claim 18, wherein the fluid passing porous material comprises an adsorbent material adapted to permit passage of the patient's perspiration at a predetermined diffusion rate.

20. The transdermal patch of claim 19, wherein the adsorbent material is selected from the group consisting of silica, silica gel, alumina, cellulose, and combinations thereof.

21. The transdermal patch of claim 1, wherein the first and second regions of the matrix are adapted to absorb perspiration from the patient's skin several times the weight of said matrix.

22. The transdermal patch of claim 21, wherein at least one of the first and second regions of the matrix is selected from the group consisting of guar, acacia, xantham gums, and combinations thereof.

23. The transdermal patch of claim 22, wherein at least one of the first and second regions of the matrix is composed of a gelling agent.

24. The transdermal patch of claim 23, wherein the gelling agent is selected from the group consisting of carboxypolymethylene, hydroxyethylcellulose, polyacrylamide, and combinations thereof.

25. The transdermal patch of claim 1, wherein the agonist is an opioid agonist.

26. The transdermal patch of claim 25, wherein the opioid agonist is selected from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propoxyphene, sufentanil, tilidine, tramadol, combinations thereof, and salts thereof.

27. The transdermal patch of claim 1, wherein the antagonist is an opioid antagonist.

28. The transdermal patch of claim 27, wherein the opioid antagonist is selected from the group consisting of naltrexone, nalmefene, cyclazacine, levallorphan, and combinations thereof.

29. A transdermal patch for administrating an agonist to a patient, said transdermal patch comprising:
a) an occlusive wall defining a reservoir with an open bottom end and an opposing top end;
b) a matrix permeable to perspiration from the patient occupying said reservoir, said matrix comprising:
a first region having the agonist suspended therein for release through the bottom end of the reservoir to the patient; and
a second region located at the top end of the reservoir, said second region having an antagonist associated with the agonist suspended therein and being configured to release the antagonist at the onset of imminent overdose of the agonist by the patient;
c) a permeable adhesive layer covering at least a portion of said bottom end of the reservoir, said adhesive layer being adapted for maintaining the matrix in communication with the skin of the patient; and
d) at least one visual indicator located at the top end of the reservoir, said at least one visual indicator being adapted to undergo a visual change upon contact with the perspiration from the patient.

30. The transdermal patch of claim 29, wherein the first and second regions of the matrix are each adapted to facilitate the diffusion of perspiration therethrough at a predetermined diffusion rate.

31. The transdermal patch of claim 30, wherein the perspiration comprises a member selected from the group consisting of ions, electrolytes, water, moisture, secretions, and combinations thereof.

32. The transdermal patch of claim 31, wherein the at least one visual indicator is a color indicator adapted to irreversibly change color upon contact with the perspiration from the patient.

33. The transdermal patch of claim 32, further including a plurality of visual indicators.

34. The transdermal patch of claim 33, wherein a first one of said plurality of visual indicators is a primary visual indicator located proximate the top end of the reservoir in communication with the second region of the matrix, said primary visual indicator being adapted to undergo a visual change to indicate delivery of the complete dose of the agonist to the patient.

35. The transdermal patch of claim 34, further comprising at least one fluid permeable column each having a visual indicator located at one end, and an inlet at the other end proximate the bottom end of the reservoir for receiving the patient's perspiration.

36. The transdermal patch of claim 35, further comprising:
first, second and third ones of said plurality of visual indicators;
a first fluid permeable column in operative association with said first visual indicator, said first fluid permeable column being adapted for facilitating the flow of perspiration from a patient to said first visual indicator at high diffusion rate relative to the matrix, wherein the visual change in the first visual indicator is indicative of the proper bonding of the adhesive layer to the patient's skin;
a second fluid permeable column in operative association with said second visual indicator, said second fluid permeable column being adapted for facilitating the flow of perspiration from a patient to said second visual indicator at a similar diffusion rate relative to the matrix, wherein the visual change in the second visual indicator is indicative of the peak delivery of the agonist to the patient; and
a third fluid permeable column in operative association with said third visual indicator, said third fluid permeable column being adapted for facilitating the flow of perspiration from a patient to said third visual indicator at a lower diffusion rate relative to the matrix, wherein the visual change in third visual indicator is indicative of the threshold at which the patient is in danger of receiving an overdose of the agonist.

37. The transdermal patch of claim 35, wherein the at least one fluid permeable column comprises a fluid passing porous material.

38. The transdermal patch of claim 37, wherein the fluid passing porous material comprises an adsorbent material adapted to permit passage of the patient's perspiration at a predetermined diffusion rate.

39. The transdermal patch of claim 38, wherein the adsorbent material is selected from the group consisting of silica, silica gel, alumina, cellulose, and combinations thereof.

40. A transdermal patch for administrating an agonist to a patient, said transdermal patch comprising:
a) an occlusive wall defining a reservoir with a bottom end and an opposing top end;
b) a matrix permeable to perspiration from the patient occupying said reservoir, said matrix comprising:
a first region having the agonist suspended therein for release through the bottom end of the reservoir to the patient; and
a second region located at the top end of the reservoir, said second region having an antagonist associated with the agonist suspended therein and being configured to release the antagonist at the onset of imminent overdose of the agonist by the patient;
c) a permeable adhesive layer covering at least a portion of said bottom end of the reservoir, said adhesive layer being adapted for maintaining the matrix in communication with the skin of the patient; and
d) first, second, third and fourth visual indicators each located at the top end of the reservoir, said first, second, third and fourth visual indicators each being adapted to undergo a visual color change upon contact with the perspiration from the patient.

41. The transdermal patch of claim 40, wherein the fourth visual indicator is located proximate the top end of the reservoir in communication with the second region of the matrix, said fourth visual indicator being adapted to undergo a visual color change to indicate delivery of the complete dose of the agonist to the patient.

42. The transdermal patch of claim 41, further comprising:
a first fluid permeable column with the first visual indicator located at one end thereof, and a first inlet at the other end proximate the bottom end of the reservoir for receiving the patient's perspiration;
a second fluid permeable column with the second visual indicator located at one end thereof, and a second inlet at the other end thereof proximate the bottom end of the reservoir for receiving the patient's perspiration; and
a third fluid permeable column with the third visual indicator located at one end thereof, and a third inlet at the other end thereof proximate the bottom end of the reservoir for receiving the patient's perspiration.

43. The transdermal patch of claim 42, wherein:
said first fluid permeable column being adapted for facilitating the flow of perspiration from a patient to said first visual indicator at high diffusion rate relative to the matrix, wherein the visual color change in the first visual indicator is indicative of the proper bonding of the adhesive layer to the patient's skin;
said second fluid permeable column being adapted for facilitating the flow of perspiration from a patient to said second visual indicator at a similar diffusion rate relative to the matrix, wherein the visual color change in the second visual indicator is indicative of the peak delivery of the agonist to the patient; and
said third fluid permeable column being adapted for facilitating the flow of perspiration from a patient to said third visual indicator at a lower diffusion rate relative to the matrix, wherein the visual color change in third visual indicator is indicative of the threshold at which the patient is in danger of receiving an overdose of the agonist.

44. The transdermal patch of claim 43, wherein the first, second and third fluid permeable columns each comprise a fluid passing porous material.

45. The transdermal patch of claim 44, wherein the fluid passing porous material comprises an adsorbent material adapted to permit passage of the patient's perspiration at a predetermined diffusion rate.

46. The transdermal patch of claim 45, wherein the adsorbent material is selected from the group consisting of silica, silica gel, alumina, cellulose, and combinations thereof.
